(19) United States
(12) Patent Application Publication
(10) Pub. No.: US 2024/0029848 A1
(43) Pub. Date: Jan. 25, 2024

(54) SYSTEMS AND METHODS FOR GENERATING A TEXT REPORT AND SIMULATING HEALTH CARE JOURNEY

(71) Applicant: Pangaea Data Limited, London (GB)

(72) Inventors: Vibhor Gupta, London (GB); Jingqing Zhang, London (GB); Julia Ive, London (GB)

(21) Appl. No.: 18/357,212

(22) Filed: Jul. 24, 2023

(30) Foreign Application Priority Data
Jul. 22, 2022 (WO) PCT/IB2022/056810

(51) Int. Cl. G16H 15/00; G16H 10/60; G16H 50/20; G16H 50/30; G06F 40/40

(52) U.S. Cl. CPC G16H 15/00; G16H 10/60; G16H 50/20; G16H 50/30; G06F 40/40

(57) ABSTRACT

A computer implemented method for generating a text report includes receiving input data including at least one table, using a first generative model to identify one or more variables in the input data and generate a table extract comprising the identified variables in a specified order, and using a second generative model to generate a text report based on the table extract, the text report including each of the extracted variables in the specified order. A system for simulating healthcare journey of one or more patients is also disclosed. The system receives a patient data; creates a simulation model of the patients; executes the simulation model to predict health variables; generates treatment variables; provides the predicted health variables, the treatment variables and clinician inputs to the simulation model for continuous learning of the simulation model.

### FIELD OF THE INVENTION

This invention relates in general to the field of data processing using machine learning and deep learning models and, in particular, to the generation of text reports from input data. More particularly, the invention relates to simulation of healthcare journey of patients.

### BACKGROUND OF THE INVENTION

Data to text generation, such as the generation of text reports based on tabulated input data, is a widely studied problem with a range of approaches and applications in the field of industrial reporting, including e.g. financial, medical and general scientific reporting.

Current text generation models may be based on neural networks pre-trained using a significant amount of data. Current models require a lot of data for proper training, however, such an amount of data is rarely available in practice. In certain fields in particular there may be a problem of data scarcity, limiting the availability of training data, and as a result models struggle to produce accurate and interpretable text reports.

The accumulation of healthcare data has recently reached unprecedented levels. The collection of this data is critical for research and development in the medical domain, such as developing new drugs, treatments or therapies; understanding the characteristics and comorbidities of rare diseases; and techniques for the earlier detection of certain diseases. For all these use cases, it is critical to identify the target patients first at scale, either for recruitment into studies, or a deeper analysis of the data of patients and diseases.

A commonly used method to identify such target patients is to use medical coding systems like International Classification of Diseases (ICD). However, several studies have highlighted flaws in their real-world application which make them unsuitable for classifying patients, especially with rare and/or highly specific diseases in real practice. A study conducted to evaluate the inter-rater agreement between two coders on the same patient's health record concludes that ICD is not reliable in primary care. Another large-scale survey from different countries reported that the majority of the respondents found that ICD codes had no clustering mechanisms, lacked specificity, had no terms for describing complications or adverse events, and had vague term definitions. A more recent study evaluated the accuracy of ICD-9-CM codes in determining the genotypes for Sickle Cell Disease for healthcare quality studies, and found that ICD codes displayed an accuracy as low as 23% for certain SCD genotypes, making them unsuitable for research purposes. A similar study for Crohn's disease and diabetes analysed the accuracy and completeness of ICD codes in ambulatory visits by analysing the coding performed by 23 clinicians and found that over 25% of the appropriate codes were not recorded and omitted, leading to an incorrect and incomplete characterisation of the patients' conditions. ICD codes are also prone to miscoding as a recent study conducted in the context of pulmonary embolisms found that up to 18% of patients coded with ICD-10 were false positives, and 9% were false negatives. The authors attributed these errors to the vague documentation for physicians responsible for assigning codes. Overall, as the ICD codes may not be reliable for a number of diseases and manual inspection of thousands of or even millions of patients by clinical experts is highly costly and time-consuming, an automatic pipeline with machine learning and specific expert guidelines can be useful to standardise, scale-up and accelerate the identification of patients with rare and complex diseases.

Moreover, electronic health records (EHRs) have proved to be a key to increasing of quality care of patients. The EHRs include a range of data including demographics, medical history, medication and allergies, laboratory test results, clinical notes etc. These EHRs when provided to doctors, clinicians or medical researchers, use them to identify disease trajectory and treatment for patients. However, for this process they rely on medical coding systems such as International Classification of Diseases (ICD) to identify patients with specific diseases from Electronic Health Records (EHRs). However, due to the lack of detail and specificity as well as a probability of miscoding, recent studies suggest the ICD codes often cannot characterise patients accurately for specific diseases in real clinical practice, and as a result, using them to find patients for studies or trials can result in high failure rates and missing out on uncoded patients. Manual inspection of all patients at scale is not feasible as it is highly costly and slow.

Furthermore, clinical notes contain information not present elsewhere, including drug response and symptoms, all of which are highly important when predicting key outcomes in acute care patients. The conventional methods do not predict outcomes of a patient based on the clinical notes.

The present invention aims to address these problems in the state of the art.

### SUMMARY OF THE INVENTION

The present disclosure seeks to provide a method and system for generating a text report. The present disclosure provides a computer-readable medium comprising instructions which when executed by a processor, cause the processor to perform the method for generating a text report. An aim of the present disclosure is to provide a simulation model for simulating healthcare journey of one or more patients. An aim of the present disclosure is to provide a solution that overcomes at least partially the problems encountered in prior art and provides a system that simulates the healthcare journey of one or more patients to accurately predict the patient outcome.

In one aspect, an embodiment of the present disclosure provides a computer implemented method for generating a text report, the method comprising:

receiving input data comprising at least one table, wherein the input data is received by one or more processors configured to operate individually or in concert;

using a first generative model to identify one or more variables in the input data, wherein identifying the one or more variables includes extracting one or more labels for rows and/or columns of the at least one table and generate a table extract comprising the identified one or more variables in a specified order, wherein generating the table extract includes determining a location of each of the identified one or more variables in the input data and copying each of the identified one or more variables directly from the input data to the table extract; and

using a second generative model to generate a text report based on the table extract, the text report including each of the extracted one or more variables in the specified order, wherein the second generative model is configured to generate one or more paragraphs of text including at least each of the extracted one or more variables in the specified order defined by the table extract.

In another aspect, an embodiment of the present disclosure provides a computer-readable medium comprising instructions which when executed by a processor, cause the processor to perform the aforesaid method.

In yet another aspect, an embodiment of the present disclosure provides a data processing system for generating a text report, comprising:

a first generative model configured to:
receive input data comprising at least one table, wherein the input data is received by one or more processors configured to operate individually or in concert;
identify one or more variables in the input data, wherein identifying the one or more variables includes extracting one or more labels for rows and/or columns of the at least one table, and
generate a table extract comprising the identified one or more variables in a specified order, wherein generating the table extract includes determining a location of each of the identified one or more variables in the input data and copying each of the identified one or more variables directly from the input data to the table extract; and

a second generative model configured to:
generate a text report based on the table extract, the text report including each of the extracted one or more variables in the specified order, wherein the second generative model is configured to generate one or more paragraphs of text including at least each of the extracted one or more variables in the specified order defined by the table extract.

In yet another aspect, an embodiment of the present disclosure provides a method of training the data processing system, the method comprising:

training the first generative model using first training data which comprises a plurality of tables and associated table extracts; and

training the second generative model using second training data which comprises a plurality of reports and associated table extracts.

In yet another aspect, an embodiment of the present disclosure provides a system for simulating healthcare journey of one or more patients, wherein the system comprises:

a database configured to store a patient data related to the one or more patients;

a processor configured to:
receive a patient data stored in the database and/or from an external source;
create a simulation model of the one or more patients using the received patient data by employing machine learning;
execute the simulation model to predict one or more health variables;
generate one or more treatment variables in response to the generated one or more health variables;
provide the predicted one or more health variables, the one or more treatment variables and one or more clinician inputs to the simulation model for continuous learning of the simulation model.

In yet another aspect, an embodiment of the present disclosure provides a method for simulating healthcare journey of one or more patients, the method comprising:

storing a patient data related to the one or more patients in a database;

receiving a patient data stored in the database and/or from an external source;

creating a simulation model of the one or more patients using the received patient data by employing machine learning;

executing the simulation model to predict one or more health variables;

generating one or more treatment variables in response to the generated one or more health variables;

providing the predicted one or more health variables, the one or more treatment variables and one or more clinician inputs to the simulation model for continuous learning of the simulation model.

In yet another aspect, an embodiment of the present disclosure provides the simulation model of the one or more patients created by the processor, wherein the processor is further configured to:

receive a raw data related to a plurality of diseases, from the database;

prepare an unstructured textual data and a structured data from the raw data;

extract the one or more health variables from the unstructured textual data, using a pre-trained algorithm;

combine the one or more health variables from the unstructured textual data with the structured data to obtain aggregated features;

feed the aggregated features and the one or more clinician inputs to the machine learning to build a disease classifier;

apply the disease classifier to the patient data of one or more patients.

In yet another aspect, an embodiment of the present disclosure provides the method for creating the simulation model of the one or more patients, comprising:

receiving a raw data related to a plurality of diseases, from the database;

preparing an unstructured textual data and a structured data from the raw data;

extracting the one or more health variables from the unstructured textual data, using a pre-trained algorithm;

combining the one or more health variables from the unstructured textual data with the structured data to obtain aggregated features;

feeding the aggregated features and the one or more clinician inputs to the machine learning for building a disease classifier;

applying the disease classifier to the patient data of one or more patients.

Embodiments of the present disclosure substantially eliminate or at least partially address the aforementioned problems in the prior art, and enables automated generation of text reports and provides a system and method for simulating healthcare journey of the one or more patients.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

For a better understanding of the present invention and to show more clearly how it may be carried into effect, reference will now be made by way of example only, to the accompanying drawings, in which:

FIG. 1 is schematic diagram showing a processing system according to an embodiment;

FIG. 2 is an illustration showing an example text report generation process;

FIG. 3 is a flowchart showing a method according to an embodiment; and

FIG. 4 is a flowchart showing a method of training a data processing system according to an embodiment.

FIG. 5 illustrates a system for simulating healthcare journey of one or more patients.

FIG. 6 is flowchart showing a method for simulating healthcare journey of one or more patients.

FIG. 7 is a flowchart showing a method of creating a simulation model of the one or more patients.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a computer implemented method for generating a text report, the method comprising:

receiving input data comprising at least one table, wherein the input data is received by one or more processors configured to operate individually or in concert;

using a first generative model to identify one or more variables in the input data, wherein identifying the one or more variables includes extracting one or more labels for rows and/or columns of the at least one table and generate a table extract comprising the identified one or more variables in a specified order, wherein generating the table extract includes determining a location of each of the identified one or more variables in the input data and copying each of the identified one or more variables directly from the input data to the table extract; and

using a second generative model to generate a text report based on the table extract, the text report including each of the extracted one or more variables in the specified order, wherein the second generative model is configured to generate one or more paragraphs of text including at least each of the extracted one or more variables in the specified order defined by the table extract.

In another aspect, an embodiment of the present disclosure provides a computer-readable medium comprising instructions which when executed by a processor, cause the processor to perform the aforesaid method.

In yet another aspect, an embodiment of the present disclosure provides a data processing system for generating a text report, comprising:

a first generative model configured to:
receive input data comprising at least one table, wherein the input data is received by one or more processors configured to operate individually or in concert;
identify one or more variables in the input data, wherein identifying the one or more variables includes extracting one or more labels for rows and/or columns of the at least one table, and
generate a table extract comprising the identified one or more variables in a specified order, wherein generating the table extract includes determining a location of each of the identified one or more variables in the input data and copying each of the identified one or more variables directly from the input data to the table extract; and

a second generative model configured to:
generate a text report based on the table extract, the text report including each of the extracted one or more variables in the specified order, wherein the second generative model is configured to generate one or more paragraphs of text including at least each of the extracted one or more variables in the specified order defined by the table extract.

In yet another aspect, an embodiment of the present disclosure provides a method of training the data processing system, the method comprising:

training the first generative model using first training data which comprises a plurality of tables and associated table extracts; and

training the second generative model using second training data which comprises a plurality of reports and associated table extracts.

In yet another aspect, an embodiment of the present disclosure provides a system for simulating healthcare journey of one or more patients, wherein the system comprises:

a database configured to store a patient data related to the one or more patients;

a processor configured to:
receive a patient data stored in the database and/or from an external source;
create a simulation model of the one or more patients using the received patient data by employing machine learning;
execute the simulation model to predict one or more health variables;
generate one or more treatment variables in response to the generated one or more health variables;
provide the predicted one or more health variables, the one or more treatment variables and one or more clinician inputs to the simulation model for continuous learning of the simulation model.

In yet another aspect, an embodiment of the present disclosure provides a method for simulating healthcare journey of one or more patients, the method comprising:

storing a patient data related to the one or more patients in a database;

receiving a patient data stored in the database and/or from an external source;

creating a simulation model of the one or more patients using the received patient data by employing machine learning;

executing the simulation model to predict one or more health variables;

generating one or more treatment variables in response to the generated one or more health variables;

providing the predicted one or more health variables, the one or more treatment variables and one or more clinician inputs to the simulation model for continuous learning of the simulation model.

In yet another aspect, an embodiment of the present disclosure provides the simulation model of the one or more patients created by the processor, wherein the processor is further configured to:

receive a raw data related to a plurality of diseases, from the database;

prepare an unstructured textual data and a structured data from the raw data;

extract the one or more health variables from the unstructured textual data, using a pre-trained algorithm;

combine the one or more health variables from the unstructured textual data with the structured data to obtain aggregated features;

feed the aggregated features and the one or more clinician inputs to the machine learning to build a disease classifier;

apply the disease classifier to the patient data of one or more patients.

In yet another aspect, an embodiment of the present disclosure provides the method for creating the simulation model of the one or more patients, comprising:

receiving a raw data related to a plurality of diseases, from the database;

preparing an unstructured textual data and a structured data from the raw data;

extracting the one or more health variables from the unstructured textual data, using a pre-trained algorithm;

combining the one or more health variables from the unstructured textual data with the structured data to obtain aggregated features;

feeding the aggregated features and the one or more clinician inputs to the machine learning for building a disease classifier;

applying the disease classifier to the patient data of one or more patients.

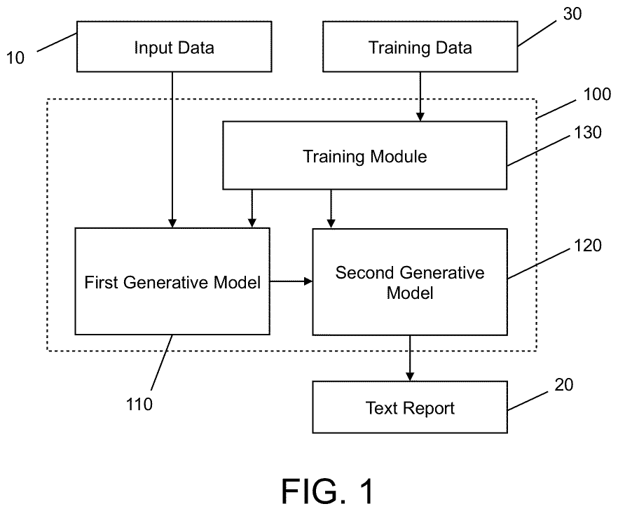

FIG. 1 of the accompanying drawings shows a schematic diagram of an embodiment of a processing apparatus 100 according to the present invention. The processing apparatus 100 is configured to receive input data 10 and output a text report 20. The processing apparatus 100 may comprise one or more processors configured to operate individually or in concert. The processing apparatus 100 comprises a first generative model 110, a second generative model 120, and a training module 130. Each of the component modules of the processing apparatus 100 may be a software module. Each may be implemented separately on one or more processors or jointly on a singular set of one or more processors.

The first generative model 110 is configured to receive input data 10 comprising at least one table. The input data 10 may comprise scientific data in the form of one or more tables. For example, the input data 10 may be experimental data e.g. medical data collected during one or more experiments. In other examples, the input data 10 may be data sourced from any suitable field e.g. industrial, engineering, scientific or financial data, population census or opinion survey data, or data automatically extracted by parsing or “crawling” one or more libraries of information, e.g. the internet.

In some examples, the at least one table in the input data 10 is a two dimensional array. Each entry in the array may contain one or more data objects such as, for example, variables, parameters, values, measurements, units, results, labels, classifications etc. One or more of the entries of the array may include a label applicable to the data object contained in one or more other related entries. For example, a label in one entry may define a number, unit, named entity, column name, row name, variable, category, parameter, classification etc. applicable at least one other related entry. Examples of such labels may include “Time (s)”, “Length (m)”, Efficacy (%), “Result”, “Total”, “Drug Name” etc. The related entries may therefore include values which are defined by the aforementioned labels. In some examples, labels may be provided in an entry at or near the start of a row or column, where the related entries include some or all of the remaining entries in the row or column. In some examples, such labels may be provided in a legend accompanying the table.

In some embodiments, the processing apparatus 100 may be configured to flatten the at least one table by reading the table row-by-row or column-by-column. In this way, the two dimensional array of each table is reduced to a one-dimensional array. By flattening the table, the method can improve the ability of the first generative model 110 to parse the input data 10, improving the processing efficiency. This further allows the processing apparatus 100 to be implemented with models which may require or function more effectively with such input data e.g. seq2seq models.

The first generative model 110 is configured to identify one or more variables in the input data 10 and generate a table extract comprising the identified variables in a specified order. The first generative model 110 may be a seq2seq, which means sequence-to-sequence model. For example, the first generative model 110 may be based on a transformer based architecture, a long short-term memory, LSTM, architecture, or any other suitable seq2seq model.

Variables in the input data 10 may be parameters which can have a range of values. The range of values may be bounded or unbound, and may be continuous or discretised. Each variable may be defined by a label or variable name which defines the values and provides a defined meaning for the values. In some examples, a label for one or more variables may be explicitly included in an entry of the array. Alternatively, the label or variable name may be implicit, or separately provided, e.g. in a legend.

In some embodiments, the first generative model 110 may be configured to identify the one or more variables by extracting one or more labels for rows and/or columns. In some examples, labels may be provided in an entry at or near the start of a row or column, where the related entries include some or all of the remaining entries in the row or column. In some examples, such labels may be provided in a legend accompanying the table.

By extracting labels, the first generative model 110 can ensure that the table extract includes all the necessary details required to fully describe the input data 10 in the table. In many cases, each value in an input data table is associated with a variable label. In some examples, each row may be associated with a different variable. In such examples, a row label may be provided in an entry at or near the start of each row. Each column may be associated with a dataset label defining e.g. time points or experiments associated with each value. Alternatively, each column may be associated with a different variable and each row may be associated with a dataset label. In some embodiments, the first generative model 110 may be configured to identify which of the rows or columns includes labels which correspond to variables.

In some examples, the table extract may be a text string including each of the variables in the specified order. Alternatively, the table extract may be implemented with any suitable data structure. The specified order may be determined by the first generative model 110 based on the training data. In some examples, the specified order may include each of the identified variables in order, followed by each of the associated data sets. In some examples, one or more of the variables and/or data sets may be grouped together, e.g. dataset corresponding to a common outcome. In this way, conventions in how data is commonly discussed in reports can be inferred from the training data and applied to the text report generation process.

In some embodiments, first generative model 110 may be configured to generate the table extract by determining a location of each identified variable in the input data 10 and copying each identified variable directly from the input data 10 to the table extract. In this way, the first generative model 110 can avoid errors in the text generation process by implementing a copy mechanism which copies each variable directly from the input table to the table extract. In some examples, the copy mechanism may be integrated with the first generative model 110. For example, the copy mechanism may be implemented as an extra layer, sigmoid and loss function introduced into the first generative model 110.

The second generative model 120 is configured to generate a text report based on the table extract. The text report 20 includes each of the extracted variables in the specified order. The second generative model 120 may be a seq2seq model. For example, the second generative model 120 may be based on a transformer based architecture, a long short-term memory, LSTM, architecture, or any other suitable seq2seq model.

The generated text report 20 may be one or more paragraphs of text including at least each of the extracted variables in the specified order defined by the table extract. The second generative model 120 may be configured to generated complete sentence discussing each variable in a clear manner. The specified order may follow conventions inferred from the training data and applied by the first generative model 110.

In this way, the processing apparatus 100 is capable of generating one or more paragraphs of text which accurately summarise the input data 10 provided in table form. In some examples, the processing apparatus 100 is capable of generating scientific reports, e.g. medical reports, from tabular scientific data. In this way, the processing apparatus 100 can ease the burden of a technical expert, e.g. a doctor, tasked with writing a report to summarise the findings in their data, saving the time of these technical experts which is highly valuable and which can be more efficiently applied to the task of generating more data. Moreover, by applying the described two-model approach, the processing apparatus 100 is capable of maximising both the extractive and abstractive potential of text generation. That is, the processing apparatus 100 can ensure that all of the necessary details of the input data 10 are correctly extracted, and further ensure that each of the extracted details is explained clearly in the output text report 20.

In some embodiments, a second generative model 120 may be configured to generate the text report 20 further based on contextual information for the at least one table. For example, contextual information may be any of a title, legend, footnote and/or caption of the table. By taking account of contextual information not within the table itself, the second generative model 120 can ensure that the data in the input table is explained clearly in the text report 20. For example, a table caption may provide an indication of the conclusions that can be reached based on the data in the table.

In some embodiments, a second generative model 120 may be configured to generate the text report 20 further based on one or more specified rows and/or columns of the at least one table. The specified rows and/or columns may be e.g. the last row or the last column. In some examples, the last 1 to 3 rows of a table may be considered. In many tables, the last row or rows may include a summary or result value based on the preceding rows e.g. a total value, an average value, ora classification/outcome/conclusion label. For example, the last row of a table may indicate a result or outcome for each of a plurality of experiments or datasets. Alternatively, or in addition, such values may be found in a last column or columns. By basing the text report 20 on such values specifically, the second generative model 120 can ensure that the correct summary or result is explained for each variable included in the table extract.

In this way, by providing the second generative model 120 with the table extract and, optionally, on or both of the contextual information and/or specified rows/columns, the amount of data to be processed by the second generative model 120 can be controlled. Alternatively, or in addition, the second generative model 120 may be configured to generate the text report 20 further based on the table in its entirety. In some examples, the full table may be provided to the second generative model 120 instead of or in addition to the contextual information and/or specified rows/columns discussed above. In this way, the second generative model 120 may be provided with all of the available values, and of which may be indicative of specific language to be used in the generated text report 20.

In some embodiments, each of the first generative model 110 and the second generative model 120 may be based on the same seq2seq model architecture. The model architecture may be a transformer based architecture, a long short-term memory, LSTM, architecture, or any other suitable seq2seq model. In this way, the implementation of the processing apparatus 100 can be greatly simplified. By implementing a common architecture for the first and second generative models 110,120, it is possible to unify the hardware and processing requirements for both components of the processing apparatus 100. In some examples, the two models can be trained in a complementary manner, in that the models utilise the same form of training data and training process and so a common training process for both models can be implemented. Alternatively, in some embodiments a different architecture may be used for each of the generative models.

The training module 130 is configured to train the processing apparatus 100. In particular, the training module is configured to train the first generative model 110 and the second generative model 120. In some embodiments, the training module 130 may be separate from the processing apparatus 100, for example, the training module 130 may be implemented in a separate device which is connected to the processing module 100 for the purpose of training the processing apparatus 100, and subsequently disconnected.

The training module 30 is configured to receive training data 30. The received training data 30 may be suitable to training one or both of the first and second generative models 110,120 directly. For example, the training data 30 may include first training data for training the first generative model 110 and second training data for training the second generative model 120. In some examples, the first training data may include a plurality of tables and associated table extracts, and the second training data includes a plurality of reports and associated table extracts.

In some embodiments, the training data 30 may include associated tables, table extracts and reports. The training module 130 may be confirmed to generate the first and second training data by separating the training data 30 appropriately.

In some embodiments, the training module 130 may be configured to receive training data 30 comprising a plurality of example reports, each comprising at least one table. In some examples, the reports may be scientific reports e.g. medical reports. In some examples, the reports may be related to industrial, scientific, financial, engineering, social, marketing or big data reporting and analysis. Any suitable reports may be used to train the models for a specific task or context, where the report includes at least one table and the text of the report includes one or more sentences relating to the data shown in the table.

The training module 130 may be configured to generate first training data for training the first generative model 110 and second training data for training the second generative model 120, using the received example reports. In some examples, the first training data may include a plurality of tables and associated table extracts, and the second training data includes a plurality of reports and associated table extracts.

In some embodiments, the training module 130 may be configured to generate the first and second training data by extracting at least one table from each received example report, and parsing the report for one or more labels found in the table. One or more of the entries of the array may include a label applicable to the data object contained in one or more other related entries. For example, a label in one entry may define a number, unit, named entity, column name, row name, variable, category, parameter, classification etc. applicable to at least one other related entry. Examples of such labels may include “Time (s)”, “Length (m)”, Efficacy (%), “Result”, “Total”, “Drug Name” etc. The related entries may therefore include values which are defined by the aforementioned labels. In some examples, labels may be provided in an entry at or near the start of a row or column, where the related entries include some or all of the remaining entries in the row or column. In some examples, such labels may be provided in a legend accompanying the table.

The training module 130 may be configured to identify one or more labels in the text of the report. The training module 130 may be configured to delete the rest of the report to obtain a table extract. The first training data may be formed using each extracted table and table extract, and the second training data may be formed using each report and table extract.

In this way, each single training data example, i.e. each report, in the received training data 30 can be processed to generate two associated examples in the first training data set and the second training data set. In this way, the training module 130 can directly generate the training data needed to train each of the generative models independently.

In some embodiments, the training module 130 may be configured to generate additional training data by detecting one or more values in each table and/or report and creating new training data by replacing the detected values with one or more modified values.

In some embodiments, the values may be detected based on a library e.g. the training data itself, an annotated subset of the training data, or a larger corpus of related subject matter. For example, where the training data 30 includes a plurality of medical reports, a dictionary which includes biomedical concepts (e.g. drug name, body weight, units) and numeric values (e.g. 10 mg/kg, 50 kg) may be collected based on a larger external corpus in the medical/scientific field (e.g. PubMed scientific literature. Based on the dictionary, the values may be detected in the reports in the training data 30 and converted to placeholders, and their corresponding entries in tables may also be converted to placeholders.

In some examples, placeholders of values may be replaced by other values in the dictionary, e.g. values under the same concept such as other body weight values. In some examples, placeholders of values may be replaced by applying randomization, for example, by selecting a value within variation range, e.g. 80%-110%, of the original values. A larger or smaller range may be selected according to the dataset. The type of values, e.g. integers or floats, and an associated precision, may be maintained.

Alternatively, or in addition, the training module 130 may be configured to generate additional training data by detecting one or more variables in each table and/or report and creating new training data by replacing the detected variables with one or more synonyms or similar words.

In some embodiments, the variables may be detected based on a library, as described above. In some examples, the placeholders of variables may be replaced by other variables, e.g. corresponding to biomedical concepts, in the dictionary. In some examples, placeholders of variables may be replaced by applying randomization to certain alphanumeric variables, for example, changing a drug name from ABCD123 to BCDE234. In some example, placeholders of variables may be replaced by searching for one or more synonyms or similarly categorised terms in an external resource, e.g. an external word replacement tool.

In this way, the training module 130 can improve the training of the respective generative models by augmenting the received training data 30. In particular, the data augmentation operating can improve the text reports generated when the volume of the received training data 30 is low. Such ‘low-resource’ situations are common in scientific technical fields, e.g. medical reporting, where the number of suitable reports available for training data may be very low, e.g. fewer than 200 samples.

Alternatively, in some embodiments, the models may be trained without prior augmentation of the training data 30 e.g. if the amount of training data 30 received is sufficient.

In some embodiments, the training module 130 may be configured to pre-train each of the first generative model 110 and the second generative model 120 using a larger volume of general training data.

The larger volume of training data may comprise, for example, a larger external library of related data, e.g. for generating medical reports, a larger corpus of PubMed scientific literature may be used to pre-train the models. In this way, the training module 130 can take advantage of general pre-training based on the language of the field, and reserve the specific training data for fine tuning the models. In this way, the training module 130 can maximise the use of the received training data 30 which may be limited in supply. Alternatively, in some embodiments the models may be trained directly on the received training data 30, without pre-training.

In some embodiments, the training module 130 may be configured to train the first generative model 110 using the first training data which comprises the plurality of tables and associated table extracts.

In some embodiments, the training module 130 may be configured to train the second generative model 120 using the second training data which comprises the plurality of reports and associated table extracts.

In some embodiments, the second generative model 120 may also be trained using the one or more tables associated with each report in the second training data. Alternatively, or in addition, the second generative model 120 may also be trained using contextual information for the at least one table. For example, contextual information may be any of a title, legend, footnote and/or caption of the table. Alternatively, or in addition, the second generative model 120 may also be trained using one or more specified rows and/or columns of the at least one table. The specified rows and/or columns may be e.g. the last row or the last column. In some examples, the last 1 to 3 rows of a table may be considered.

In this way, by providing the second generative model 120 with the table extract and, optionally, on or both of the contextual information and/or specified rows/columns, the amount of data used to train the second generative model 120 can be controlled, and in some examples may be linked to the form of data to be processed by the second generative model 120 in practice.

By training each of the models individually in this way, the training module 130 can ensure, in particular, that the generated table extract is accurate and suitable for generating a report which both (a) includes all of the necessary information from the table, and (b) clearly explains the information and conclusions associated with the table. In this way, the training module 130 can prevent one or more downsides associated with an end to end training method.

For example, an underfitting problem may arise where the training data produces suitable reports but inaccuracies are introduced using real world data. This problem is especially common where the set of received training data is small. In an end to end training process, such errors may arise in part due to the generation of an unsuitable or inaccurate table extract, which is essentially a ‘hidden variable’. As such, by training in two stages the training module 130 can validate the generation of the table extract and reduce inaccuracies due to underfitting. Performance and accuracy can be especially improved in situations where the set of available training data is small. Optionally, the data processing system employs a Generative Large-Language-Models (G-LLMs) for generating the text report. The Generative Large-Language-Models (G-LLMs) are deep learning algorithms trained on a large amount of data.

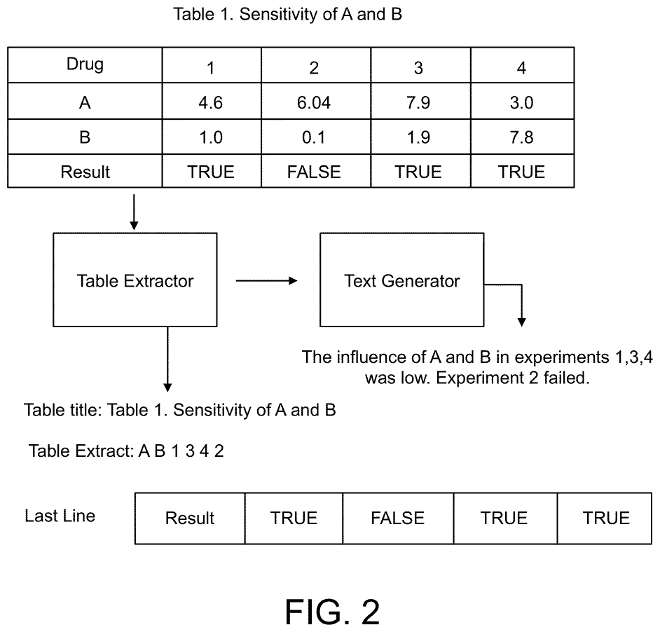

FIG. 2 shows an illustration of an exemplary text report generation process by the processing apparatus 100 of FIG. 1.

The processing apparatus 100 receives input data 10 in the form of the table “Table 1” shown below:

TABLE 1
Sensitivity of A and B

| Drug | 1 | 2 | 3 | 4 |
|---|---|---|---|---|
| A | 4.6 | 6.04 | 7.9 | 3.0 |
| B | 1.0 | 0.1 | 1.9 | 7.8 |
| Result | TRUE | FALSE | TRUE | TRUE |

The first generative model 110, also referred to as a “Table Extractor” receives the input data 10 comprising the table above. As shown, the table in the input data 10 is a two dimensional array. Each entry in the array contains a data object including, for example, variables, measurements, results and labels. One or more of the entries of the array includes a label applicable to the data object contained in one or more other related entries. For example, the labels “A” and “B” in the first column define a drug name variable and a row name applicable to their related entries. These labels are provided in entries at the start of their respective rows, and the related entries include the remaining entries in each row. The labels “1”, “2”, “3” and “4” in the first row define an experiment number variable and a column name applicable to their related entries. These labels are provided in entries at the start of their respective columns, and the related entries include the remaining entries in each column.

The first generative model 110 identifies one or more variables in the input data 10 and generates a table extract comprising the identified variables in a specified order. As shown, the first generative model 110 identifies the one or more variables by extracting one or more labels for rows and/or columns. The labels are provided in an entry at or near the start of a row or column, where the related entries include some or all of the remaining entries in the row or column. As shown, the first generative model 110 identifies the variables “A”, “B”, “1”, “2”, “3” and “4”.

The specified order is determined by the first generative model 110 based on the training data. As shown, the specified order may include the identified drug name variables for each of the rows in order, followed by each identified experiment number variable for each of the columns. As shown, the experiment number variables are grouped together to collect datasets corresponding to a common outcome.

The first generative model 110 generates the table extract by determining a location of each identified variable in the input data 10 and copying each identified variable directly from the input data 10 to the table extract. The first generative model 110 outputs the table extract as follows:
“Table extract: A B 1 3 4 2”

The second generative model also referred to as a “Text Generator” 120 generates a text report 20 based on the table extract.

The text report 20 is further based on contextual information for the received table. As shown, the contextual information includes a title “Table 1. Sensitivity of A and B”. The text report 20 is further based on a specified row of the received table. As shown, the specified row is the last line of the table. The last row includes a result value based on the preceding rows, i.e. an outcome label for each experiment.

The text report 20 includes each of the extracted variables in the specified order. As shown, the generated text report 20 may be one or more paragraphs of text including at least each of the extracted variables in the specified order defined by the table extract. The second generative model 120 generates a complete sentence discussing each variable in a clear manner. The specified order follows conventions inferred from the training data as applied by the first generative model 110. The processing apparatus 100 outputs a text report 20 as follows:

“The influence of A and B in the experiments 1, 3, 4 was low. Experiment 2 failed.” Optionally, a third model also referred to as a “Auto-corrector” corrects the text generated by the text generator. Beneficially, the auto-corrector reduces errors in the generated text and thus improves the model performance.

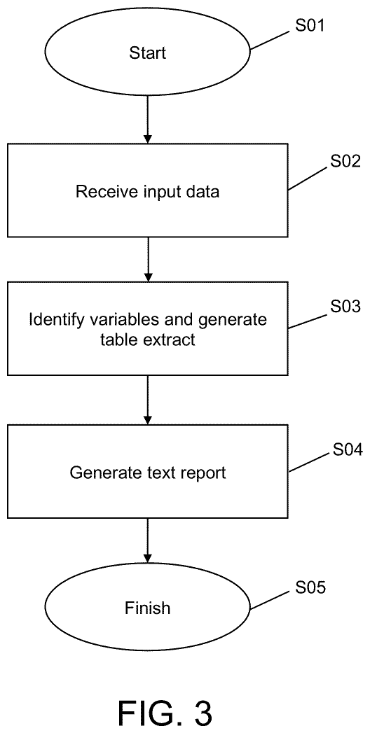

FIG. 3 of the accompanying drawings shows a flowchart representing a method of generating a text report according to an embodiment. The method starts at step S01.

At step S02, the method includes receiving input data comprising at least one table. The input data may be received by one or more processors configured to operate individually or in concert. The input data may comprise scientific data in the form of one or more tables. For example, the input data may be experimental data e.g. medical data collected during one or more experiments. In other examples, the input data may be data sourced from any suitable field e.g. industrial, engineering, scientific or financial data, population census or opinion survey data, or data automatically extracted by parsing or “crawling” one or more libraries of information, e.g. the internet.

In some examples, the at least one table in the input data is a two dimensional array. Each entry in the array may contain one or more data objects such as, for example, variables, parameters, values, measurements, units, results, labels, classifications etc. One or more of the entries of the array may include a label applicable to the data object contained in one or more other related entries. For example, a label in one entry may define a number, unit, named entity, column name, row name, variable, category, parameter, classification etc. applicable to at least one other related entry. Examples of such labels may include “Time (s)”, “Length (m)”, Efficacy (%), “Result”, “Total”, “Drug Name” etc. The related entries may therefore include values which are defined by the aforementioned labels. In some examples, labels may be provided in an entry at or near the start of a row or column, where the related entries include some or all of the remaining entries in the row or column. In some examples, such labels may be provided in a legend accompanying the table.

In some embodiments, the method may include flattening the at least one table by reading the table row-by-row or column-by-column. In this way, the two dimensional array of each table is reduced to a one-dimensional array. By flattening the table, the method can improve the ability of the first generate model to parse the input data, improving the processing efficiency. This further allows the method to be implemented with models which may require or function more effectively with such input data e.g. seq2seq models.

At step S03, the method includes using a first generative model to identify one or more variables in the input data and generate a table extract comprising the identified variables in a specified order. The first generative model may be a seq2seq model. For example, the first generative model may be based on a transformer based architecture, a long short-term memory, LSTM, architecture, or any other suitable seq2seq model.

Variables in the input data may be parameters which can have a range of values. The range of values may be bounded or unbound, and may be continuous or discretised. Each variable may be defined by a label or variable name which defines the values and provides a defined meaning for the values. In some examples, a label for one or more variables may be explicitly included in an entry of the array. Alternatively, the label or variable name may be implicit, or separately provided, e.g. in a legend.

In some embodiments, identifying the one or more variables includes extracting one or more labels for rows and/or columns. In some examples, labels may be provided in an entry at or near the start of a row or column, where the related entries include some or all of the remaining entries in the row or column. In some examples, such labels may be provided in a legend accompanying the table.

By extracting labels, the method can ensure that the table extract includes all of the necessary details required to fully describe the input data in the table. In many cases, each value in an input data table is associated with a variable label. In some examples, each row may be associated with a different variable. In such examples, a row label may be provided in an entry at or near the start of each row. Each column may be associated with a dataset label defining e.g. time points or experiments associated with each value. Alternatively, each column may be associated with a different variable and each row may be associated with a dataset label. In some embodiments, the method may include identifying which of the rows or columns includes labels which correspond to variables.

In some examples, the table extract may be a text string including each of the variables in the specified order. Alternatively, the table extract may be implemented with any suitable data structure. The specified order may be determined by the first generative model based on the training data. In some examples, the specified order may include each of the identified variables in order, followed by each of the associated data sets. In some examples, one or more of the variables and/or data sets may be grouped together, e.g. dataset corresponding to a common outcome. In this way, conventions in how data is commonly discussed in reports can be inferred from the training data and applied to the text report generation process.

In some embodiments, generating the table extract may include determining a location of each identified variable in the input data and copying each identified variable directly from the input data to the table extract. In this way, the method can avoid errors in the text generation process by implementing a copy mechanism which copies each variable directly from the input table to the table extract. In some examples, the copy mechanism may be integrated with the first generative model. For example, the copy mechanism may be implemented as an extra layer, sigmoid and loss function introduced into the first generative model.

At step S04, the method includes using a second generative model to generate a text report based on the table extract. The text report includes each of the extracted variables in the specified order. The second generative model may be a seq2seq model. For example, the second generative model may be based on a transformer based architecture, a long short-term memory, LSTM, architecture, or any other suitable seq2seq model.

The generated text report may be one or more paragraphs of text including at least each of the extracted variables in the specified order defined by the table extract. The second generative model may be configured to generate a complete sentence discussing each variable in a clear manner. The specified order may follow conventions inferred from the training data and applied by the first generative model.

In this way, the method is capable of generating one or more paragraphs of text which accurately summarise the input data in table form. In some examples, the method is capable of generating scientific reports, e.g. medical reports, from tabular scientific data. In this way, the method can ease the burden of a technical expert, e.g. a doctor, tasked with writing a report to summarise the findings in their data, saving the time of these technical experts which is highly valuable and which can be more efficiently applied to the task of generating more data. Moreover, by applying the described two-model approach, the method is capable of maximising both the extractive and abstractive potential of text generation. That is, the method can ensure that all of the necessary details of the input data are correctly extracted, and further ensure that each of the extracted details is explained clearly in the output text report.

In some embodiments, generating the text report may be further based on contextual information for the at least one table. For example, contextual information may be any of a title, legend, footnote and/or caption of the table. By taking account of contextual information not within the table itself, the second generative model can ensure that the data in the input table is explained clearly in the text report. For example, a table caption may provide an indication of the conclusions that can be reached based on the data in the table.

In some embodiments, generating the text report may be further based on one or more specified rows and/or columns of the at least one table. The specified rows and/or columns may be e.g. the last row or the last column. In some examples, the last 1 to 3 rows of a table may be considered. In many tables, the last row or rows may include a summary or result value based on the preceding rows e.g. a total value, an average value, or a classification/outcome/conclusion label. For example, the last row of a table may indicate a result or outcome for each of a plurality of experiments or datasets. Alternatively or in addition, such values may be found in a last column or columns. By basing the text report on such values specifically, the method can ensure that the correct summary or result is explained for each variable included in the table extract.

In this way, by providing the second generative model with the table extract and, optionally, one or both of the contextual information and/or specified rows/columns, the amount of data to be processed by the second generative model can be controlled. Alternatively, or in addition, generating the text report may be further based on the table in its entirety. In some examples, the full table may be provided to the second generative model instead of or in addition to the contextual information and/or specified rows/columns discussed above. In this way, the second generative model may be provided with all of the available values, and of which may be indicative of specific language to be used in the generated text report.

In some embodiments, each of the first generative model and the second generative model may be based on the same seq2seq model architecture. The model architecture may be a transformer based architecture, a long short-term memory, LSTM, architecture, or any other suitable seq2seq model. In this way, the implementation of the method can be greatly simplified. By implementing a common architecture for the first and second generative models, it is possible to unify the hardware and processing requirements for both stages of the method. In some examples, the two models can be trained in a complementary manner, in that the models utilise the same form of training data and training process and so a common training process for both models can be implemented. Alternatively, in some embodiments a different architecture may be used for each of the generative models.

The method finishes step at S05.

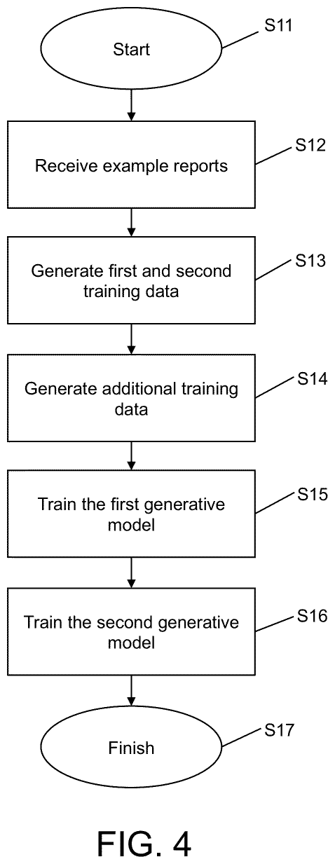

FIG. 4 of the accompanying drawings shows a flowchart representing a method of training a data processing system according to an embodiment. The method of FIG. 4 may be used to train the data processing system described with respect to FIG. 1. The method starts at step S11.

At step S12, the method includes receiving a plurality of example reports, each comprising at least one table. In some examples, the reports may be scientific reports e.g. medical reports. In some examples, the reports may be related to industrial, scientific, financial, engineering, social, marketing or big data reporting and analysis. Any suitable reports may be used to train the models for a specific task or context, where the report includes at least one table and the text of the report includes one or more sentences relating to the data shown in the table.

Alternatively, in some embodiments, the method may include receiving pre-prepared training data suitable to training one or both of the first and second generative models directly.

At step S13, the method includes generating first training data for training the first generative model and second training data for training the second generative model. In some examples, the first training data may include a plurality of tables and associated table extracts, and the second training data includes a plurality of reports and associated table extracts.

In some embodiments, generating the first and second training data may include extracting at least one table from each received example report, and parsing the report for one or more labels found in the table. One or more of the entries of the array may include a label applicable to the data object contained in one or more other related entries. For example, a label in one entry may define a number, unit, named entity, column name, row name, variable, category, parameter, classification etc. applicable to at least one other related entry. Examples of such labels may include “Time (s)”, “Length (m)”, Efficacy (%), “Result”, “Total”, “Drug Name” etc. The related entries may therefore include values which are defined by the aforementioned labels. In some examples, labels may be provided in an entry at or near the start of a row or column, where the related entries include some or all of the remaining entries in the row or column. In some examples, such labels may be provided in a legend accompanying the table.

The method may include identifying one or more labels in the text of the report. The method may include deleting the rest of the report to obtain a table extract. The first training data may be formed using each extracted table and table extract, and the second training data may be formed using each report and table extract.

In this way, each single training data example, i.e. each report, can be processed to generate two associated examples in the first training data set and the second training data set. In this way, the method can directly generate the training data needed to train each of the generative models independently.

Alternatively, in some embodiments, pre-prepared training data may be received comprising associated tables, table extracts and reports, wherein generating the first and second training data may include separating the training data appropriately. Alternatively, pre-prepared training data may be received in appropriate sets of first and second training data suitable for training the first and second generative models respectively.

At step S14, the method may include generating additional training data by detecting one or more values in each table and/or report and creating new training data by replacing the detected values with one or more modified values.

In some embodiments, the values may be detected based on a library e.g. the training data itself, an annotated subset of the training data, or a larger corpus of related subject matter. For example, where the training data includes a plurality of medical reports, a dictionary which includes biomedical concepts (e.g. drug name, body weight, units) and numeric values (e.g. 10 mg/kg, 50 kg) may be collected based on a larger external corpus in the medical/scientific field (e.g. PubMed scientific literature). Based on the dictionary, the values may be detected in the training data reports and converted to placeholders, and their corresponding entries in tables may also be converted to placeholders.

In some examples, placeholders of values may be replaced by other values in the dictionary, e.g. values under the same concept such as other body weight values. In some examples, placeholders of values may be replaced by applying randomization, for example, by selecting a value within variation range, e.g. 80%-110%, of the original values. A larger or smaller range may be selected according to the dataset. The type of values, e.g. integers or floats, and an associated precision, may be maintained.

Alternatively, or in addition, the method may include generating additional training data by detecting one or more variables in each table and/or report and creating new training data by replacing the detected variables with one or more synonyms or similar words.

In some embodiments, the variables may be detected based on a library, as described above. In some examples, the placeholders of variables may be replaced by other variables, e.g. corresponding to biomedical concepts, in the dictionary. In some examples, placeholders of variables may be replaced by applying randomization to certain alphanumeric variables, for example, changing a drug name from ABCD123 to BCDE234. In some example, placeholders of variables may be replaced by searching for one or more synonyms or similarly categorised terms in an external resource, e.g. an external word replacement tool.

In this way, the method can improve the training of the respective generative models by augmenting the available training data. In particular, the data augmentation operating can improve the text reports generated when the availability of training data is low. Such ‘low-resource’ situations are common in scientific technical fields, e.g. medical reporting, where the number of suitable reports available for training data may be very low, e.g. fewer than 200 samples. Alternatively, in some embodiments, the models may be trained without prior augmentation of the training data e.g. if the amount of training data available is sufficient.

In some embodiments, the method may include pre-training each of the first generative model and the second generative model using a larger volume of general training data.

The larger volume of training data may comprise, for example, a larger external library of related data, e.g. for generating medical reports, a larger corpus of PubMed scientific literature may be used to pre-train the models. In this way, the method can take advantage of general pre-training based on the language of the field, and reserve the specific training data for fine tuning the models. In this way, the method can maximise the use of the available training data which may be limited in supply. Alternatively, in some embodiments the models may be trained directly on the received training data, without pre-training.

At step S15, the method includes training the first generative model using the first training data which comprises the plurality of tables and associated table extracts.

At step S16, the method includes training the second generative model using the second training data which comprises the plurality of reports and associated table extracts.

In some embodiments, the second generative model may also be trained using the one or more tables associated with each report in the second training data. Alternatively, or in addition, the second generative model may also be trained using contextual information for the at least one table. For example, contextual information may be any of a title, legend, footnote and/or caption of the table. Alternatively, or in addition, the second generative model may also be trained using one or more specified rows and/or columns of the at least one table. The specified rows and/or columns may be e.g. the last row or the last column. In some examples, the last 1 to 3 rows of a table may be considered.

In this way, by providing the second generative model with the table extract and, optionally, one or both of the contextual information and/or specified rows/columns, the amount of data used to train the second generative model can be controlled, and in some examples may be linked to the form of data to be processed by the second generative model in practice.

By training each of the models individually in this way, the method can ensure, in particular, that the generated table extract is accurate and suitable for generating a report which both (a) includes all of the necessary information from the table, and (b) clearly explains the information and conclusions associated with the table. In this way, the training method can prevent one or more downsides associated with an end to end training method.

For example, an underfitting problem may arise where the training data produces suitable reports but inaccuracies are introduced using real world data. This problem is especially common where the set of available training data is small. In an end to end training process, such errors may arise in part due to the generation of an unsuitable or inaccurate table extract, which is essentially a ‘hidden variable’. As such, by training in two stages the method can validate the generation of the table extract and reduce inaccuracies due to underfitting. Performance and accuracy can be especially improved in situations where the set of available training data is small.

The method finishes step at S17.

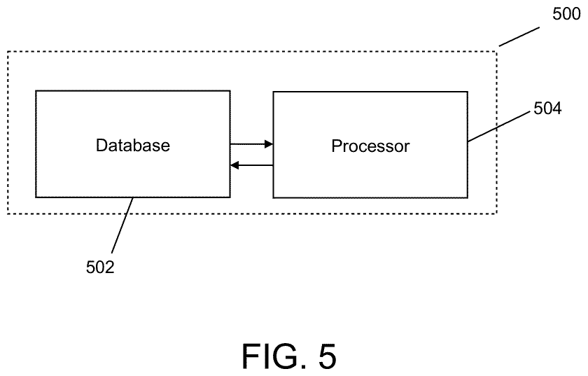

FIG. 5 of the present disclosure illustrates a system for simulating healthcare journey of one or more patients. The system 500 comprises a database 502 and a processor 504 communicably coupled with a communication network.

The system 500 comprises the database 502 configured to store patient data related to the one or more patients. The system 500 comprises the processor 504 configured to receive a patient data wherein the patient data is accessed from the database 502 and/or from an external source. The external source may include the one or more patients, wherein the external source provides the patient data via a patient input using a user-interface. The processor 504 is further configured to create a simulation model of the one or more patients, using the received patient data, and employing a machine learning. The simulation model is then executed by the processor to predict one more health variables. In response to the one or more health variables, one or more treatment variables are generated. The processor 504 provides the predicted one or more health variables, the generated one or more treatment variables and one or more clinician inputs to the simulation model for continuous learning of the simulation model. Furthermore, a final outcome including patient's healthcare journey and patient's disease diagnosis and treatment is provided to the one or more patient by the simulation model.

Throughout the disclosure, the term ‘database’ refers to an organized body of data regardless of a manner in which the data or the organized body thereof is represented. The database arrangement may comprise one or more databases, wherein the one or more databases store the data therein. Optionally, the database includes first and second database. Moreover, optionally, the database arrangement may further comprise a database management system, wherein the database management system is operable to manage the one or more databases in the database arrangement. Optionally, the database arrangement may be hardware, software, firmware and/or any combination thereof. More optionally, the data in the database arrangement may be in the form of, for example, a table, a map, a grid, a packet, a datagram, a file, a document, and a list. Moreover, the database arrangement may include data storage software and/or systems, such as, for example, a relational database like IBM DB2 and Oracle 9. Pursuant to embodiments of the present disclosure, the database stores a patient data related to the one or more patients.

Throughout the present disclosure, the term “processor” or the “processing arrangement” refers to a computational element that is operable to respond to and processes instructions that drive the system. Optionally, the processing arrangement includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “processing arrangement” may refer to one or more individual processors, processing devices and various elements associated with a processing device that may be shared by other processing devices. Additionally, the one or more individual processors, processing devices and elements are arranged in various architectures for responding to and processing the instructions that drive the system. Optionally, the processing arrangement is implemented as a remote server.

The processing arrangement is communicably coupled to the database arrangement. In this regard, the processing arrangement may be communicably coupled to the database arrangement using a data communication network. Moreover, the term “data communication network” refers to individual networks, or a collection thereof interconnected with each other and functioning as a single large network. Optionally, such a data communication network is implemented by way of wired communication network, wireless communication network, or a combination thereof. It will be appreciated that physical connection is established for implementing the wired network, whereas the wireless network is implemented using electromagnetic waves. Examples of such data communication network include, but are not limited to, Local Area Networks (LANs), Wide Area Networks (WANs), Metropolitan Area Networks (MANs), Wireless LANs (WLANs), Wireless WANs (WWANs), Wireless MANs (WMANs), the Internet, second generation (2G) telecommunication networks, third generation (3G) telecommunication networks, fourth generation (4G) telecommunication networks, fifth generation (5G) telecommunication networks, Worldwide Interoperability for Microwave Access (WiMAX), and different generation of Wireless access (Wi-Fi a, b, an, ac, ax) networks.

Throughout the disclosure, the term ‘external source’ refers to a patient himself/herself who provides the patient data via a user input device or a clinician who provides the patient data via a user input device. The user input device can be, for example, a tablet, a mobile, a computer, USB etc. The external source may provide patient data via a user input device by providing an input in the form of handwritten letters and notes.

Optionally, the patient data comprises at least one of: imaging data, genomics, clinical notes, a disease symptoms data and a plurality of lab test reports of the patient. Throughout the disclosure, the term ‘patient data’ refers to information relating to the medical or health condition of the patient. Optionally, the patient data may comprise information like demographics of the patient, information relating to the past and current health or illness, the treatment history, lifestyle choices and genetic data of the patient. Optionally, the patient data comprises of physical characteristics like biometrics that can measured by a machine or a computer. The patient data can be received from a database. Optionally, the patient data can be received from an external source that includes a patient who provides the patient data via a user input device.

Throughout the disclosure, the term ‘simulation model’ refers to a digital prototype, also known as avatar, for digitally depicting a scenario and outcome of a physical world. In general terms, simulation modeling, is the process of creating and analyzing a digital prototype of a physical model to predict its performance in the real world. For example, when a patient data is provided to the simulation model, it predicts the healthcare journey and treatment. According to the present disclosure, the simulation model, employs one or more clinician in the loop to predict the healthcare journey and treatment of the one or more patients. In an embodiment, the simulation model, is an application or software in an electronic device. The simulation model receives the patient data and provides the predicted patient outcome, patient healthcare journey and/or treatment to the patients. The simulation model is executed to predict one or more health variables, based on the patient data. The simulation model generates one or more treatment variables in response to the predicted one or more health variables. The simulation model also receives the one or more clinician inputs for continuous learning of the simulation model. The simulation model employs the pre-trained natural language processing algorithm, automated machine learning, and a disease classifier.

Optionally, the one or more health variables includes phenotypic features and patient information that comprises at least one of: demographics, response to treatments, disease outcome, social habits, family history, treatment, biomarkers, therapy medications and adverse events for the one or more patients extracted from the unstructured textual data. Throughout the disclosure, the term ‘health variables’ refers to health parameters that are measured based on the patient data. Beneficially, the one or more health variables are used to determine at least one of: eligible patients for treatment and trials, patients at risk of specific diseases, predicted patient outcomes, predicted risk of recurrence and suggestions of monitoring steps.

Optionally, the one or more treatment variables comprises at least one of: a precision medicine and the one or more clinician inputs for the one or more patients. Throughout the disclosure, the term ‘treatment variables’ refers to treatment methods suggested by the simulation model and the clinician input. Beneficially, the one or more treatment variables are used to determine improved patient outcomes and an information on recurrent patients.

Throughout the disclosure, the term ‘clinician inputs’ refer to treatment methods and suggestions given by at least one of a clinician, plurality of clinicians specialized in different medical care, one or more clinicians, nurse, doctor, specialist, medical expert, based on their expertise and knowledge. The clinician input may be provided via a user interface. In an embodiment, the eligible patients for treatment and trials, patients at risk of specific diseases, predicted patient outcomes, predicted risk of recurrence and suggestions of monitoring steps are used to determine the clinician inputs.

In accordance with the present disclosure, a system of creating the simulation model of the one or more patients is disclosed. The simulation model is created by the processor, wherein the processor is configured to receive a raw data related to a plurality of diseases, from a database. The raw data comprises a structured data and an unstructured textual data. The processor is configured to prepare an unstructured textual data and structured data from the raw data. One or more health variables from the unstructured textual data are extracted using a pre-trained algorithm. The pre-trained algorithm is a natural language processing algorithm. Optionally, the natural language programming algorithm can be a phenotypic NLP algorithm. Optionally, the one or more health variables refer to the phenotypic features of the patients. The processor is configured to combine the one or more health variables from the unstructured textual data with the structured data to obtain aggregated features of the patient. The aggregated features and the one or more clinician inputs are feed to the machine learning to build a disease classifier. The disease classifier is applied to the patient data of one or more patients to simulate healthcare journey of the one or more patients.

Advantageously, the present disclosure relates to a scalable workflow which leverages both structured data and unstructured textual notes from raw data with techniques including Natural Language Processing (for example for phenotyping), AutoML (automated machine learning) and Clinician-in-the-Loop (using one or more clinician inputs) mechanism to build machine learning classifiers (disease classifier) to identify patients at scale with given diseases. The disclosed system can more accurately identify patients with specific diseases than simply using ICD codes.

Throughout the disclosure, the term ‘raw data’ refers to electronic health records (EHRs) that are publicly available, for example, via the internet. The raw data comprises of structured data and unstructured textual data.

Optionally, the unstructured textual data comprises at least one of: nursing notes, prescriptions, insurance claims, clinical notes, discharge summaries, radiology reports. Throughout the disclosure, the term ‘unstructured textual data’ refers to textual data stored not in a specific structured format. Examples of human-generated unstructured textual data are text files, email, etc. The machine-generated unstructured textual data includes medical reports, medical data and many more. The phenotypic features extracted from unstructured textual notes are beneficial for better accuracy and interpretability of classifiers.

Optionally, the structured data comprises at least one of: blood test results, genomic testing, laboratory data, demographics, temperature, heart rate. Throughout the disclosure, the term ‘structured data’ refers to a standardized format for providing information about a page and classifying the page content; for example, on a clinical note, what are the patient health details, temperature, the blood pressure, weight, and so on.

Throughout the disclosure, the term ‘phenotypic features’ refers to the observable physical properties of an organism; these include the organism's appearance, development, and behavior. An organism's phenotype is determined by its genotype, which is the set of genes the organism carries, as well as by environmental influences upon these genes. The organism is referred to as human beings or patients in particular.

Optionally, the pre-trained algorithm is a Natural Language Processing algorithm. To leverage the rich clinical information from unstructured textual data, the system employs a pre-trained algorithm that is a Natural Language Processing (NLP) algorithm to extract one or more health variables of patients. Optionally, the NLP algorithm is a phenotypic NLP algorithm. Optionally the one or more health variables are the phenotypic features of patients. The one or more health variables are then enriched with the clinical features from structured data for predictive modelling by Automated Machine Learning (AutoML) to classify the patients. Optionally, the clinical features can be extracted from either the structured data, the unstructured data, or a combination of the structured and unstructured data. Subsequently, a clinical signature is formed by the extracted clinical features along with their clinical and statistical significance. The clinical significance is defined by the clinical guidelines and clinical knowledge base via clinician-in-the-loop mechanism. The statistical significance is determined automatically using Automated Machine Learning (AutoML). The unstructured clinical notes, such as discharge summaries, nursing notes and radiology reports, are rich in phenotype information as the clinicians naturally describe phenotypic abnormalities of patients in the narratives of notes. Leveraging the phenotype information is considered to improve the understanding of disease diagnosis, disease pathogenesis, patient outcomes and genomic diagnostics and subsequently, the automatic phenotype annotation from clinical notes has become an important task in clinical Natural Language Processing (NLP).

Optionally, the pre-trained algorithm uses Human Phenotype Ontology (HPO). In an example, the HPO standardizes over 15,000 phenotype concepts and leverages self-supervised pre-training techniques, contextualized word embeddings by the Transformer model and data augmentation techniques (paraphrasing and synthetic text generation) to capture names (like hypertension), synonyms (like high blood pressure), abbreviations (like HTN) and, more importantly, contextual synonyms of phenotypes. For example, descriptive phrases like “rise in blood pressure”, “blood pressure is increasing” and “BP of 140/90” are considered as contextual synonyms of Hypertension (HP:0000822) and finding such contextual synonyms require an understanding of context semantics. As a result of the contextual detection of phenotype, the phenotyping NLP algorithm demonstrates superior performance to baseline phenotyping algorithms including Clinphen, NCBO, cTAKES, MetaMap, MetaMapLite, NCR, MedCAT and fine-tuned BERT-based models.

Throughout the disclosure, the term ‘aggregated features’ refers to features obtained when the clinical features from structured data are combined with the extracted one or more health variables of the unstructured textual data. In an embodiment the one or more health variables are the phenotypes of the patients. The list of phenotypes that are extracted from unstructured clinical notes by the NLP algorithm are combined with the structured clinical features together as the aggregated features of patients. For instance, the aggregated features of a patient may have a list of phenotypes like weight loss (HP:0001824) and malnutrition (HP:0004395) and structured clinical features like temperature 36.6 and heart rate 86. The aggregated clinical features of patients will be then used as input features in the subsequent step which leverages Automated Machine Learning (AutoML) and Clinician-in-the-Loop mechanism to build a disease classifier on a training set and evaluate the disease classifier on a testing set. Optionally, the clinical signature is used to evaluate the disease classifier. Further, patients with a target disease are identified based on the clinical signature. The system leverages both the clinician-in-the-loop mechanism and Automated Machine Learning (AutoML) to identify significance of clinical features and the new insights derived from the clinical signatures.

Optionally, the system, based on the clinical features, generates a knowledge graph representing a strong correlation between the clinical features for a target disease. Healthcare workers, such as doctors or clinicians, can identify the presence of the target disease in a patient based on the generated knowledge graph. For example, for patients at risk of Chronic Kidney Disease (CKD), the clinical features such as elevated serum creatinine, heart failure, renal insufficiency are more significantly relevant in finding at risk CKD patients, while other features like diabetes and hypertension are relevant but less significant. Further, Cirrhosis has been found relevant with Cancer Cachexia which is new and clinically valid insights for clinicians.

Optionally, the system employs Generative Large-Language-Models (G-LLMs) for generating responses on the queries received from the clinicians. The Generative Large-Language-Models (G-LLMs) are deep learning algorithms trained on a large amount of data. The Generative Large-Language-Models (G-LLMs) assist the clinician at each step of patient journey and generate the summaries of patient journey. In this regard, the patient journey comprises some steps that are normally followed by the clinicians in evaluating the patients such as patient history evaluation, conducting physical examination, differential diagnosis, targeted clinical investigations, diagnosis, evaluation of diagnosis and then define a plan and monitoring the patient. For example, the Generative Large-Language-Models (G-LLMs) assist the clinicians in identifying the clinical features related to a target disease, in diagnostic evaluation or in generating a patient report based on test reports.

The system employs machine learning algorithms for building a disease classifier. The machine learning uses the aggregated features and the one or more clinician input to build the disease classifier. Specifically, the ‘machine learning algorithms’ refer to a category of algorithms employed by the processing arrangement that allows the processing arrangement to become more accurate in generating the document, without being explicitly programmed. More specifically, the machine learning algorithms are employed to artificially train the processing arrangement so as to enable the processing arrangement to automatically learn, from analyzing training dataset and improve performance from experience, without being explicitly programmed.

It will be appreciated that the machine learning algorithms employed by the processing arrangement is trained using a training dataset. Optionally, the processing arrangement may employ different types of machine learning algorithms, depending upon the training dataset employed. Typically, examples of the different types of machine learning algorithms, depending upon the training dataset employed for training the processing arrangement comprise, but are not limited to: supervised machine learning algorithms, unsupervised machine learning algorithms, semi-supervised learning algorithms, and reinforcement machine learning algorithms. In an embodiment, the machine learning comprises, but not limited to natural language processing (NLP), deep learning and computer vision. NLP is a subfield of linguistics, computer science, and artificial intelligence concerned with the interactions between computers and human language, in particular how to program computers to process and analyze large amounts of natural language data. Deep learning is a type of machine learning and artificial intelligence (AI) that enables computers to imitate the way humans gain certain types of knowledge. Computer vision is a field of computer science and in particular, artificial intelligence (AI), that enables computers and systems to derive meaningful information from digital images, videos and other visual inputs and take actions or make recommendations based on that information. Furthermore, the processing arrangement is trained by interpreting patterns in the training dataset and adjusting the machine learning algorithms accordingly to get a desired output.

In an embodiment, machine learning is an AutoML (automated machine learning). The machine learning workflow with the Clinician-in-the-Loop mechanism disclosed in the present disclosure leverages both structured data and unstructured textual data from EHRs to find (1) critical clinical features that help characterise a disease, especially for rare diseases which are not well defined and understood; (2) relevant patients meeting the precise criteria of the disease. AutoML is a framework for efficient and automated feature, classifier type, and hyper-parameter selection of machine learning classifiers. The classifiers are iteratively improved with gold diagnosis labels and feedback on features from clinicians via the Clinician-in-the-Loop mechanism. For example, the disclosed workflow is performed at least on four diseases including Ovarian Cancer, Lung Cancer, Cancer Cachexia and Lupus Nephritis and show superior performance to baseline methods disclosed in the specification below.

Throughout the disclosure, the term ‘disease classifer’ refers to a classifier that is a type of machine learning algorithm used to assign a disease to an input of patient data or raw data. In an embodiment, the disease classifiers are built with AutoML (automated machine learning) which use phenotypes as input features because the diagnosis of diseases may rely on a combination of multiple phenotypes (such as the American College of Rheumatology Classification Criteria for systemic lupus erythematosus (SLE)) even though the diseases themselves may not be explicitly mentioned in clinical notes. This also suggests that using phenotyping NLP algorithms like Clinphen24, NCBO25, cTAKES26, MetaMap and solely without classifiers is not sufficient to perform inference and identify patients with specific diseases. Advantageously, the Clinician-in-the-Loop mechanism of the invention improves the training set and incorporates clinically relevant features into disease classifiers which leads to better accuracy and interpretability of disease classifiers. The disease classifier works as a binary classifier for each specific disease which is built based on gold diagnosis labels and feedback from one or more clinicians input on the training set and then evaluated on the testing set. The testing set and the training set are explained below.

To build the disease classifier, the system employs the Automated Machine Learning (AutoML) which provides efficient and scalable solutions to build machine learning classifiers with minimal human assistance. In convention, building a machine learning model (including deep learning) involves a pipeline of steps that require human expertise including feature engineering, model selection, model architecture search, hyper-parameter tuning, model evaluation and more. Given the complexity of the steps, it is usually time-consuming to manually find the best configuration of a machine learning classifier and requires expertise from machine learning engineers. Therefore, AutoML is designed to automate the pipeline within a time budget by using strategies such as grid search, Bayesian Optimization and meta-learning. More specifically, the present system develops an AutoML framework based on the HpBandSter library46. This framework automatically searches for the most suitable classifier model from candidates including Support Vector Machine (SVM), Random Forest, Gradient Boosting, Logistic Regression, and Multi-layer Perceptron as well as their respective hyper-parameters. The AutoML framework also incorporates feature engineering from scikit-learn which, in practice, automatically selects top features from the aggregated features of patients.

Building disease classifiers by machine learning algorithms is restricted by two challenges: (1) lack of accurate and gold standard labels from medical experts to train and evaluate the models, for example, ICD codes in EHR database may not be accurate as discussed in background section and manually labelling EHRs at scale is highly costly; (2) lack of interpretability and clinical validity behind the decision made by the models. Therefore, in the present disclosure focuses to leverage Clinician-in-the-Loop mechanism, by which clinicians can iteratively work with the models and provide feedback for enhancement, to address the two challenges by collecting gold diagnosis labels for patients and validating clinically relevant features of diseases with clinicians.

In an exemplary embodiment, to obtain the training set, in the training stage, three clinicians are first requested to create gold diagnosis labels for patients with consensus. The annotation guideline instructs the clinicians to provide a binary label for each patient on whether the patient suffers from, has a recent history of, or is at risk of a specific disease or not. The labels provided by the clinicians are collected as the gold diagnosis labels, which are used as the targets to train disease classifiers by AutoML. Meanwhile, the system also iteratively requests the clinicians to validate the top features that a disease classifier relies on to identify patients. The importance of features is quantified by SHAP values which measure how much a feature positively or negatively impacts the decision of a classifier. The features that have the highest absolute SHAP values are taken as top features and sent to the clinicians to assess their clinical relevance to the disease. The clinically irrelevant features will be removed from the input features of patients while the relevant features will be retained. The removal of such statistically correlated but clinically irrelevant features can potentially help prevent overfitting and improve the interpretability of model predictions. The final disease classifier is trained after several iterations of feature validation until the model performance is stable (typically within 3 iterations in practice).

In an exemplary embodiment, in the testing stage, after the final disease classifier is obtained, it is applied to the testing set to predict the patients at high risk of the disease. In an embodiment, the testing set refers to the patient data or the raw data.

In an example, the present system uses 58,976 Electronic Health Records (EHRs) from MIMIC-III database which corresponds to 58,976 admissions from 46,520 unique patients in Intensive Care Units (ICU). Each EHR in MIMIC-III is assigned with relevant ICD-9 codes. The EHRs are randomly split into the training set with 29,637 EHRs and the testing set with 29,339 EHRs, and the workflow makes one prediction for each EHR. Optionally, the EHRs of the same unique patients will be all in either the training set or the testing set to avoid data leakage.

In an example, Experiments are conducted on four diseases including Ovarian Cancer, Lung Cancer, Cancer Cachexia, and Lupus Nephritis. The definitions of these diseases based on ICD-9 codes in this disclosure are shown in Table 2 below. The ICD-based criteria are then used as “noisy” labels for EHRs and as a baseline method to identify patients.

TABLE 2

| | ICD-9 Codes for | ICD-9 Codes for |
|---|---|---|
| Disease Name | Positive Cohort | Negative Cohort |
| Ovarian Cancer | 183.0 | Otherwise |
| Lung Cancer | any subcode of 162 | Otherwise |
| Cancer Cachexia | any subcode 140-239 and | any subcode 140-239 but |
| | (799.3 or 799.4) | not 799.3 nor 799.4 |
| Lupus Nephritis | 710.0 and any subcode of | Otherwise |
| | 580 | |

Table 2 illustrates the ICD code-based criteria to define positive and negative cohorts for each disease in the training and testing set. The Cancer Cachexia setup requires patients to have cancer as the background condition for both positive and negative cohorts.

TABLE 3

| | | | | | | | Testing Subset by | |
|---|---|---|---|---|---|---|---|---|
| | Entire Training Set | | Training Subset | | Entire Testing Set | | ICD | |
| Disease | Labels | | by Gold Labels | | by ICD Labels | | by Gold Labels | |
| Name | Positive | Negative | Positive | Negative | Positive | Negative | Positive | Negative |
| Ovarian | 43 | 29,594 | 57 | 43 | 38 | 29,301 | 52 | 48 |
| Cancer | | | | | | | | |
| Lung | 586 | 29,051 | 62 | 38 | 585 | 28,754 | 66 | 34 |
| Cancer | | | | | | | | |
| Cancer | 42 | 4,449 | 59 | 33 | 51 | 4,370 | 70 | 27 |
| Cachexia* | | | | | | | | |
| Lupus | 86 | 29,551 | 42 | 58 | 63 | 29,276 | 62 | 38 |
| Nephritis | | | | | | | | |

The number of positive and negative EHRs in the entire training set and entire testing set based on the ICD-based criteria defined by Table 2, as well as the training subset and testing subset which have gold diagnosis labels created via the Clinician-in-the-Loop mechanism. The training (testing) subset with gold labels is fully included by the entire training (testing) set. The disease classifiers are trained to differentiate the positive and negative EHRs. The number of EHRs with gold labels in the training and testing subset for Cancer Cachexia is fewer than 100 because a few patients who do not have cancer as the background condition are discarded.

Table 3 summarises the number of positive and negative EHRs in the entire training and testing set based on the ICD-based criteria which also shows that the prevalence of these diseases is low in MIMIC-III. However, the ICD codes cannot be used as the gold diagnosis labels given their multiple flaws, so the present disclosure uses the Clinician-in-the-Loop mechanism to create the gold diagnosis labels for EHRs. As manually creating large-scale gold labels on entire MIMIC-III datasets with clinicians is not feasible in practice, only small-scale EHRs can be selected for gold labelling and ideally, the selected EHRs should be relatively balanced (i.e. around half EHRs are positive for the disease and the other half are negative). In an example, to create the relatively balanced gold set, an initial yet imperfect disease classifier (a Random Forest classifier) is first trained solely based on the entire training set by using ICD codes as the learning target (i.e., positive and negative cohorts are decided by ICD criteria in Table 2). Then, 100 EHRs are selected from the training set by randomly sampling 25 EHRs from each of the following four patient groups: (1) EHRs that are predicted/labelled as positive by the initial classifier and ICD; (2) EHRs that are predicted as positive by the initial classifier but labelled as negative by ICD; (3) EHRs that are predicted as negative by the initial classifier but labelled as positive by ICD; (4) EHRs that are predicted/labelled as negative by the initial classifier and ICD. Similarly, another 100 EHRs are selected from the entire testing set by applying the same initial classifier. As opposed to selecting EHRs based on ICD codes directly, using the initial classifier with the four patient groups encourages finding EHRs that are mislabelled by ICD codes. The initial classifier is preliminary and only used to select these 200 EHRs. Next, the 200 EHRs are manually labelled via the Clinician-in-the-Loop mechanism following the annotation guideline. Table 3 also summarises the statistics of the training subset and testing subset which have gold diagnosis labels.

In an exemplary embodiment, two evaluation methods for the disease classifier are considered. The first evaluation method is to compare the disease classifier created by the disclosed workflow with baseline methods on the gold testing set. The following baseline methods are considered. (1) ICD codes: To use the inclusion and exclusion criteria based on ICD codes as defined by Table 2 to classify EHRs. (2) Structured data only: using the 17 clinical features from structured data only as patient features to AutoML without considering phenotypic features in unstructured notes. (3) Structured+NCR: To compare with other phenotyping NLP algorithms, the system aggregates the structured clinical features with the phenotypes that are extracted from clinical notes by NCR39 and then AutoML is used to build disease classifiers. (4) Structured+ClinicalBERT: this is similar to the previous baseline method but the phenotypes are extracted by ClinicalBERT which are fine-tuned for phenotyping following the work. On the gold testing set, the disclosure reports on Area Under the Curve of Receiver Operating Characteristic (AUC-ROC) and Area Under the Curve of Precision-Recall (AUC-PR) in addition to precision, recall, F1, and specificity which use 0.5 as the threshold. Importantly, AUC-ROC and AUC-PR are not applicable to ICD codes as the labels of ICD codes are discrete in binary (either 0 or 1) rather than continuous in probability.

TABLE 4
Illustrates comparison of the disease classifier created by the disclosed workflow
with baseline methods on the gold testing subset. The method with the highest
F1 score and AUC-ROC for each disease is highlighted in bold. Importantly,
all methods except ICD Codes use our AutoML to build disease classifiers.

| Disease | | | | | | AUC- | AUC- |
|---|---|---|---|---|---|---|---|
| Name | Method | Precision | Recall | F1 | Specificity | PR | ROC |
| Ovarian | ICD Codes | 0.946 | 0.714 | 0.814 | 0.957 | 0.834 | 0.836 |
| Cancer | Structured | 0.605 | 0.885 | 0.719 | 0.375 | 0.595 | 0.630 |
| | Data Only | | | | | | |
| | Structured + | 0.797 | 0.981 | 0.879 | 0.729 | 0.792 | 0.855 |
| | NCR | | | | | | |
| | Structured + | 0.788 | 1.000 | 0.881 | 0.708 | 0.788 | 0.854 |
| | ClinicalBERT | | | | | | |
| | Ours | 0.847 | 0.962 | 0.901 | 0.813 | 0.821 | 0.877 |
| Lung | ICD Codes | 0.960 | 0.727 | 0.828 | 0.941 | 0.878 | 0.834 |
| Cancer | Structured | 0.702 | 0.894 | 0.787 | 0.265 | 0.698 | 0.579 |
| | Data Only | | | | | | |
| | Structured + | 0.780 | 0.970 | 0.865 | 0.471 | 0.777 | 0.720 |
| | NCR | | | | | | |
| | Structured + | 0.800 | 0.909 | 0.851 | 0.559 | 0.787 | 0.734 |
| | ClinicalBERT | | | | | | |
| | Ours | 0.887 | 0.833 | 0.859 | 0.794 | 0.849 | 0.814 |
| Cancer | ICD Codes | 0.780 | 0.557 | 0.650 | 0.593 | 0.754 | 0.575 |
| Cachexia | Structured | 0.722 | 1.00 | 0.838 | 0 | 0.722 | 0.500 |
| | Data Only | | | | | | |
| | Structured + | 1.000 | 0.211 | 0.348 | 1.000 | 0.661 | 0.605 |
| | NCR | | | | | | |
| | Structured + | 0.857 | 0.105 | 0.188 | 0.977 | 0.600 | 0.541 |
| | ClinicalBERT | | | | | | |
| | Ours | 1.00 | 0.757 | 0.862 | 1.00 | 0.932 | 0.878 |
| Lupus | ICD Codes | 0.979 | 0.758 | 0.855 | 0.974 | 0.892 | 0.866 |
| Nephritis | Structured | 0.933 | 0.677 | 0.785 | 0.921 | 0.832 | 0.799 |
| | Data Only | | | | | | |
| | Structured + | 0.981 | 0.855 | 0.914 | 0.974 | 0.929 | 0.914 |
| | NCR | | | | | | |
| | Structured + | 0.919 | 0.919 | 0.919 | 0.868 | 0.895 | 0.894 |
| | ClinicalBERT | | | | | | |
| | Ours | 0.967 | 0.952 | 0.959 | 0.947 | 0.950 | 0.949 |

Table 4 compares the proposed workflow with the baseline methods. While the ICD codes tend to have high precision except for Cancer Cachexia, the proposed workflow achieves significantly higher recall than the ICD codes for Ovarian Cancer (0.962 vs 0.714), Lung Cancer (0.833 vs 0.727), Cancer Cachexia (0.757 vs 0.557), and Lupus Nephritis (0.952 vs 0.758), which suggests the disease classifier is more sensitive and tends to find more positive patients. This also leads to a higher F1 score of the disease classifiers for Ovarian Cancer (0.901 vs 0.814), Lung Cancer (0.859 vs 0.828), Cancer Cachexia (0.862 vs 0.650) and Lupus Nephritis (0.959 vs 0.855). In addition, the benefit of leveraging phenotypic features from unstructured notes by phenotyping NLP algorithms is also observed as the proposed workflow achieves significantly higher F1, AUC-PR and AUC-ROC than the baseline method which uses structured data only across all four diseases. Moreover, the phenotyping NLP algorithm used by the proposed workflow outperforms other phenotyping NLP algorithms, namely NCR and fine-tuned ClinicalBERT, for patient identification with consistently better AUC-ROC across four diseases.

TABLE 5
Illustrated is the comparison of the disease classifier created
by the disclosed workflow with baseline methods on the entire testing
set. Importantly, Pₑₛₜ of ICD Codes is taken from Table 4 while
Pₑₛₜ of the disclosed workflow is computed following the second
evaluation method disclosed above.

| | | | | Estimated |
|---|---|---|---|---|
| | | Number of | | Number of |
| | | Predicted | Estimated | Actual |
| Disease | | Positive EHRs | Precision | Positive EHRs |
| Name | Method | N_{pred} | Pₑₛₜ | N_{pred} × Pₑₛₜ |
| Ovarian Cancer | ICD | 38 | 0.946 | 36 |
| | Codes | | | |
| | Ours | 143 | 0.776 | 111 |
| Lung Cancer | ICD | 585 | 0.960 | 562 |
| | Codes | | | |
| | Ours | 1209 | 0.766 | 926 |
| Cachexia in | ICD | 51 | 0.780 | 40 |
| Cancer | Codes | | | |
| | Ours | 326 | 0.969 | 316 |
| Lupus | ICD | 63 | 0.979 | 62 |
| Nephritis | Codes | | | |
| | Ours | 142 | 0.758 | 108 |

Table 5 evaluates the disclosed workflow on the entire testing set which has 29,339 EHRs. The estimated precision of the proposed workflow is computed based on validation from clinicians while the estimated precision of ICD codes is taken from Table 4. For Cancer Cachexia, the proposed workflow achieves higher estimated precision and a larger number of positive predictions than the ICD codes. This leads to a significantly higher estimation of actual positive EHRs founded by the proposed workflow than the ICD codes (316 vs 40) which is 690% more EHRs. For Ovarian Cancer, Lung Cancer and Lupus Nephritis, the proposed workflow has lower estimated precision but predicts much more EHRs as positive than ICD codes, which overall lead to finding more actual positive EHRs.

Furthermore, it is seen that among the unstructured data, the phenotype has been received the least attention for the ICU monitoring. This is mainly due to the challenge to extract the phenotypic information expressed by a variety of contextual synonyms. For example, such a phenotype as Hypotension can be expressed in text as “drop in blood pressure” and “BP of 79/48”. However, the phenotypes are crucial for understanding disease diagnosis, identifying important disease-specific information, stratifying patients and identifying novel disease subtypes. The present disclosure thoroughly investigates the value of phenotypic information as extracted from text for ICU monitoring. The disclosure automatically extracts mentions of phenotypes from clinical text using a self-supervised methodology with recent advancements in clinical NLP-contextualized word embeddings that are particularly helpful for the detection of contextual synonyms. For example, extraction of those mentions for over phenotype concepts of the Human Phenotype Ontology (HPO). The phenotypic features extracted in this manner are enriched with the information coming from the structured data (i.e., bedside measurements and laboratory test results).

In an example, the disclosure uses Area Under the Curve of Receiver Operating Characteristic (AUC-ROC) and Area Under the Curve of Precision-Recall (AUC-PR) for In-Hospital Mortality and Physiological Decompensation tasks. The disclosure primarily relies on AUC-ROC for statistical analysis as it is threshold independent and used by the benchmark as the primary metric. For the Length of Stay (LOS) task, the disclosure uses Cohen's Kappa and Mean Absolute Deviation (MAD) with primarily relying on the Kappa scores for statistical analysis.

In an example, the disclosed is an investigated performance of two classifiers: Random Forest (RF) and LSTM. For each of them the following set of features are investigated: structured features only (S) and structured features enriched with phenotypic features coming from one of the three phenotype annotators (ClinicalBERT, NCR, ours). The main results are presented in Table 6. Overall, they show that phenotypic information complements positively the structured information to improve performance on all tasks. The improvements with our phenotyping model are statistically significant across all tasks compared against using structured features only or alternative phenotyping algorithms, except for In-Hospital Mortality with RF.

| Classification | | | |
|---|---|---|---|
| Model | Features Design | AUC-ROC | AUC-PR |
| SAPS-II | — | 0.756 | 0.312 |
| APACHE-III | — | 0.733 | 0.308 |
| Random Forest | S | 0.800 (0.775, | 0.339 (0.286, |
| | | 0.824) | 0.395) |
| | S + NCR | 0.828 (0.802, | 0.467 (0.404, |
| | | 0.853) | 0.529) |
| | S + CB | 0.812 (0.787, | 0.403 (0.345, |
| | | 0.838) | 0.463) |
| | S + Ours | 0.845 (0.826, | 0.462 (0.404, |
| | | 0.873) | 0.524) |
| LSTM | S⁴ | 0.825 | 0.410 |
| | S | 0.826 (0.801, | 0.391 (0.334, |
| | | 0.848) | 0.452) |
| | S + NCR | 0.841 (0.818, | 0.453 (0.393, |
| | | 0.864) | 0.513) |
| | S + CB | 0.826 (0.802, | 0.414 (0.355, |
| | | 0.849) | 0.476) |
| | S + Ours | 0.845 (0.823, | 0.464 (0.405, |
| | | 0.868) | 0.523) |

### (a) In-Hospital Mortality

### (b) Physiological Decompensation

| Classification | | | |
|---|---|---|---|
| Model | Features Design | AUC-ROC | AUC-PR |
| Random Forest | S | 0.826 (0.821, | 0.130 (0.123, |
| | | 0.831) | 0.138) |
| | S + NCR | 0.825 (0.821, | 0.124 (0.118, |
| | | 0.830) | 0.131) |
| | S + CB | 0.826 (0.821, | 0.125 (0.118, |
| | | 0.830) | 0.132) |
| | S + Ours | 0.845 (0.840, | 0.180 (0.171, |
| | | 0.850) | 0.190) |
| LSTM | S⁴ | 0.809 | 0.125 |
| | S | 0.824 (0.819, | 0.126 (0.119, |
| | | 0.829) | 0.133) |
| | S + NCR | 0.834 (0.829, | 0.134 (0.127, |
| | | 0.839) | 0.142) |
| | S + CB | 0.833 (0.828, | 0.114 (0.108, |
| | | 0.838) | 0.119) |
| | S + Ours | 0.839 (0.834, | 0.145 (0.138, |
| | | 0.844) | 0.153) |

| Classification | | | |
|---|---|---|---|
| Model | Features Design | Kappa | MAD |
| Random Forest | S | 0.390 (0.388, | 136.8 (136.2, |
| | | 0.392) | 137.4) |
| | S + NCR | 0.390 (0.388, | 142.5 (141.9, |
| | | 0.392) | 143.1) |
| | S + CB | 0.376 (0.374, | 144.3 (143.7, |
| | | 0.379) | 144.9) |
| | S + Ours | 0.420 (0.418, | 110.3 (109.3, |
| | | 0.422) | 111.3) |
| LSTM | S⁴ | 0.395 | 126.7 |
| | S | 0.380 (0.377, | 157.0 (156.3, |
| | | 0.382) | 157.6) |
| | S + NCR | 0.406 (0.404, | 123.3 (122.8, |
| | | 0.408) | 123.9) |
| | S + CB | 0.388 (0.386, | 120.1 (119.6, |
| | | 0.390) | 120.6) |
| | S + Ours | 0.430 (0.427, | 116.7 (116.1, |
| | | 0.432) | 117.2) |

### (c) Length of Stay

Illustrates results for Table 6 (a-c), (a) In-Hospital Mortality, (b) Physiological Decompensation, and (c) Length of Stay. Test set scores are shown with 95% confidence intervals in brackets if applicable. The best score for each classifier is highlighted in bold. The first row of LSTM refers to scores reported in previous literature, while the second row regards scores reproduce in this study with a comparable cohort. Here, S refers to Structured, NCR to Neural Concept Recognizer, CB to ClinicalBERT, and Ours to our phenotyping model disclosed in the present system.

The present disclosure also relates to the method as described above. Various embodiments and variants disclosed above apply mutatis mutandis to the method.

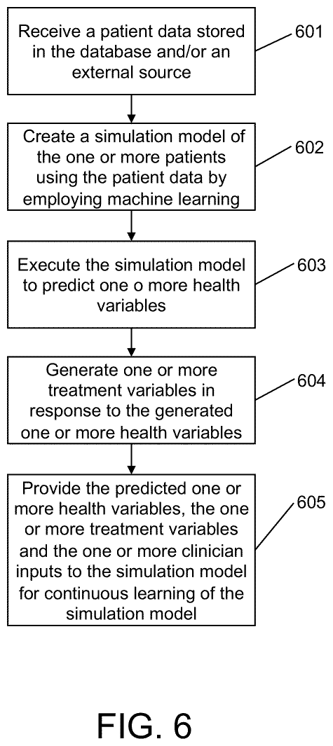

According to another embodiment of the present disclosure, a method for simulating healthcare journey of one or more patients is disclosed performing the steps 601 to 605 of FIG. 6. At step 601, a patient data is received, wherein the patient data is accessed from a database and/or from an external source like directly from the one or more patients via a patient input. At step 602, a simulation model of the one or more patients is created, using the received patient data, employing a machine learning. At step 603, the simulation model is executed to predict one more health variables. At step 604, in response to the one or more health variables, one or more treatment variables are generated. At step 605, the predicted one or more health variables, the generated one or more treatment variables and one or more clinician inputs are provided to the simulation model for continuous learning of the simulation model. Furthermore, a final outcome including patient's healthcare journey and patient's disease diagnosis and treatment is provided to the one or more patient by the simulation model.

Optionally, patient data comprises at least one of: imaging data, genomics, clinical notes, a disease symptoms data and a plurality of lab test reports of the patient.

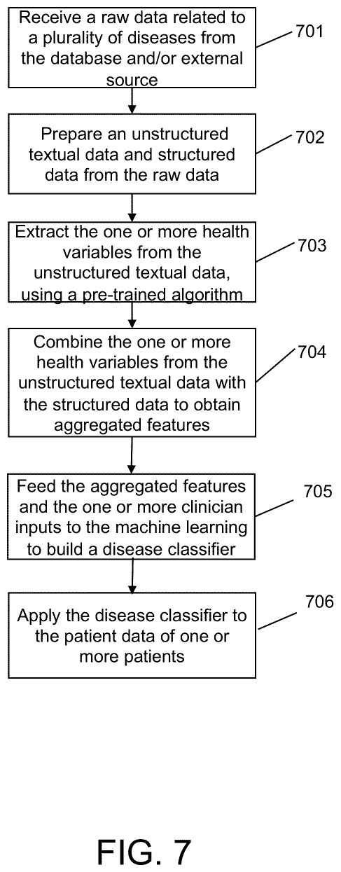

In accordance with the present disclosure, FIG. 7 illustrates the method of creating the simulation model of the one or more patients, to perform the steps 701 to 706. At step 701 a raw data related to a plurality of diseases, is received from a database. The raw data comprises of a structured data and an unstructured textual data. At step 702 an unstructured textual data and structured data are prepared from the raw data. At step 703 one or more health variables from the unstructured textual data are extracted using a pre-trained algorithm. At step 704, the one or more health variables from the unstructured textual data are combined with the structured data to obtain aggregated features of the patient. At step 705, the aggregated features and the one or more clinician inputs are feed to the machine learning to build a disease classifier. At step 706, the disease classifier is built to apply the disease classifier to the patient data of one or more patients.

Optionally, the unstructured textual data comprises at least one of: nursing notes, prescriptions, insurance claims, clinical notes, discharge summaries, radiology reports.

Optionally, the structured data comprises at least one of: blood test results, genomic testing, laboratory data, demographics, temperature, heart rate.

Optionally, the one or more health variables includes phenotypic features and patient information that comprises at least one of: demographics, adverse events, disease outcome, response to treatments, social habits, family history, treatment, biomarkers, therapy and medications, extracted from the unstructured textual data.

Optionally, the one or more health variables are used to determine at least one of: eligible patients for treatment and trials, patients at risk of specific diseases, predicted patient outcomes, predicted risk of recurrence and suggestions of monitoring steps.

Optionally, the eligible patients for treatment and trials, patients at risk of specific diseases, predicted patient outcomes, predicted risk of recurrence and suggestions of monitoring steps are used to determine the one or more clinician input.

Optionally, the one or more treatment variables comprises at least one of: a precision medicine and the one or more clinician inputs for the one or more patients.

Optionally, the one or more treatment variables are used to determine improved patient outcomes and an information on recurrent patients.

Optionally, the pre-trained algorithm is a natural language processing algorithm.

Although aspects of the invention herein have been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the scope of the invention as defined by the appended claims.

1. A system for simulating healthcare journey of one or more patients, wherein the system comprises:
a database configured to store a patient data related to the one or more patients;
a processor configured to:
receive a patient data stored in the database and/or from an external source;
create a simulation model of the one or more patients using the received patient data by employing machine learning;
execute the simulation model to predict one or more health variables;
generate one or more treatment variables in response to the generated one or more health variables;
provide the predicted one or more health variables, the one or more treatment variables and one or more clinician inputs to the simulation model for continuous learning of the simulation model.

2. The system of claim 1, wherein patient data comprises at least one of: imaging data, genomics, clinical notes, a disease symptoms data and a plurality of lab test reports of the patient.

3. The system of claim 1, wherein the simulation model of the one or more patients is created by the processor, wherein the processor is further configured to:
receive a raw data related to a plurality of diseases, from the database;
prepare an unstructured textual data and a structured data from the raw data;
extract the one or more health variables from the unstructured textual data, using a pre-trained algorithm;
combine the one or more health variables from the unstructured textual data with the structured data to obtain aggregated features;
feed the aggregated features and the one or more clinician inputs to the machine learning to build a disease classifier;
apply the disease classifier to the patient data of one or more patients.

4. The system of claim 3, wherein the unstructured textual data comprises at least one of: nursing notes, prescriptions, insurance claims, clinical notes, discharge summaries, radiology reports.

5. The system of claim 3, wherein the structured data comprises at least one of: blood test results, genomic testing, laboratory data, demographics, temperature, heart rate.

6. The system of claim 3, wherein the one or more health variables includes phenotypic features and patient information that comprises at least one of: demographics, adverse events, disease outcome, response to treatments, social habits, family history, treatment, biomarkers, therapy and medications, extracted from the unstructured textual data.

7. The system of claim 1, wherein the one or more health variables are used to determine at least one of: eligible patients for treatment and trials, patients at risk of specific diseases, predicted patient outcomes, predicted risk of recurrence and suggestions of monitoring steps.

8. The system of claim 7, wherein the eligible patients for treatment and trials, patients at risk of specific diseases, predicted patient outcomes, predicted risk of recurrence and suggestions of monitoring steps are used to determine the one or more clinician input.

9. The system of claim 3, wherein the aggregated features are obtained by combining clinical features from the structured data with the extracted one or more health variables of the unstructured textual data.

10. The system of claim 9, wherein a clinical signature of a target disease is based on the clinical features and their clinical and statistical significance with respect to the target disease.

11. The system of claim 9, wherein Generative Large Language Models (G-LLM) are employed to assist the clinicians at each step of patient journey and to generate the summaries of patient journey.

12. A method for simulating healthcare journey of one or more patients, the method comprising:
storing a patient data related to the one or more patients in a database;
receiving a patient data stored in the database and/or from an external source;
creating a simulation model of the one or more patients using the received patient data by employing machine learning;
executing the simulation model to predict one or more health variables;
generating one or more treatment variables in response to the generated one or more health variables;
providing the predicted one or more health variables, the one or more treatment variables and one or more clinician inputs to the simulation model for continuous learning of the simulation model.

13. The method of claim 12, wherein patient data comprises at least one of:
imaging data, genomics, clinical notes, a disease symptoms data and a plurality of lab test reports of the patient.

14. The method of claim 12, wherein creating the simulation model of the one or more patients, comprising:
receiving a raw data related to a plurality of diseases, from the database;
preparing an unstructured textual data and a structured data from the raw data;
extracting the one or more health variables from the unstructured textual data, using a pre-trained algorithm;
combining the one or more health variables from the unstructured textual data with the structured data to obtain aggregated features;
feeding the aggregated features and the one or more clinician inputs to the machine learning for building a disease classifier;
applying the disease classifier to the patient data of one or more patients.

15. The method of claim 14, wherein the unstructured textual data comprises at least one of: nursing notes, prescriptions, insurance claims, clinical notes, discharge summaries, radiology reports.

16. The method of claim 14, wherein the structured data comprises at least one of: blood test results, laboratory data, demographics, temperature, heart rate.

17. The method of claim 14, wherein the one or more health variables includes phenotypic features and patient information that comprises at least one of: demographics, adverse events, disease outcome, response to treatments, social habits, family history, treatment, biomarkers, therapy and medications, extracted from the unstructured textual data.

18. The method of claim 12, wherein the one or more health variables are used to determine at least one of: eligible patients for treatment and trials, patients at risk of specific diseases, predicted patient outcomes, predicted risk of recurrence and suggestions of monitoring steps.

19. The method of claim 18, wherein the eligible patients for treatment and trials, patients at risk of specific diseases, predicted patient outcomes, predicted risk of recurrence and suggestions of monitoring steps are used to determine the one or more clinician input.

20. A computer-readable medium comprising instructions which, when executed by a processor, cause the processor to perform the method of claim 12.
